(19) United States
(12) Patent Application Publication
(10) Pub. No.: US 2025/0041162 A1
(43) Pub. Date: Feb. 6, 2025

(54) CAP FOR A LOCKING ASSEMBLY OF AN ENTERAL FEEDING SYSTEM

(71) Applicant: TREMMA, Billère (FR)

(72) Inventor: Anne MERCIER, Billère (FR)

(21) Appl. No.: 18/717,498

(22) PCT Filed: Dec. 7, 2022
(86) PCT No.: PCT/EP2022/084874

§ 371 Date: Jul. 24, 2024

(30) Foreign Application Priority Data
Dec. 7, 2021 (FR) FR2113096

(51) Int. Cl. A61J 15/00

(52) U.S. Cl. CPC A61J 15/0061; A61J 15/0015

(57) ABSTRACT

One of the aims of this invention is to make available a mechanism with which it is possible to avoid situations where numerous movements made by a wearer of an enteral feeding system accidentally cause a locking assembly (200) of the enteral feeding system to change from a locked state to an unlocked state. To this end, the inventor proposes a cap which, when the locking assembly (200) is in the locked state, permits the rotational locking of the locking assembly (200). In this way, the movements made by the wearer of the enteral feeding system can no longer accidentally cause the locking assembly (200) to change from the locked state to the unlocked state.

### TECHNICAL FIELD

The invention relates to the field of connector systems for enteral feeding. In particular, it concerns a cap for a locking assembly of an enteral feeding system.

### PRIOR ART

Locking mechanisms are known for enteral feeding systems that are locked by rotation between two mating locking elements.

However, some wearers of enteral feeding systems, such as children, frequently move around when the enteral feeding system is in place, and particularly at night.

In this case, numerous movements made by the wearer of the enteral feeding system can inadvertently cause the locking assembly to change over from a locked state to an unlocked state.

Said situation may have serious consequences on the health of the wearer of the enteral feeding system.

There is therefore a need for a solution whereby the above-described situation can be prevented.

### SUMMARY OF THE INVENTION

The invention sets out at least partially to solve this need.

In particular, the invention concerns a cap for a locking assembly of an enteral feeding system.

The locking assembly in particular comprises a base of a catheter device and a connector adapted to be removably connected to the base so that the connector and the base are locked to each other in vertical and horizontal direction, the locking assembly being configured to be locked/unlocked by rotation of the connector relative to the base.

In practice, the base has a first end part, a central part and a second end part opposite the first end part, the first end part and second end part protruding from the central part, the base having an upper surface and a lower surface, the upper surface comprising a first locking element.

The connector comprises a connector body and a conduit, the conduit defining a fluid pathway and being coupled to a tube, the connector body having an upper surface and a lower surface, the lower surface comprising a second locking element of shape matching the shape of the first locking element and being configured to move between an engaging configuration and a locking configuration.

In practice, in the engaging configuration, the first locking element and the second locking element engage with each other.

In the locking configuration, in response to rotation of the connector relative to the base in a predetermined locking direction, the conduit lies above an end part of the base, called locking end part, and the first locking element and second locking element are engaged with each other so that the connector and the base are locked together in vertical and horizontal direction.

In the invention, the cap comprises a cap body in elastically deformable material which deforms under a force and returns to an initial non-deformed state on removal of the force, the cap body comprising a cap body having a top wall and a first vertical wall supporting the top wall and which is connected at a back end of the top wall.

In particular, the first vertical wall comprises a first passageway opening which extends over all or part of a width and/or height of the first vertical wall, and which:
in the engaging configuration, allows passing of the tube,
in the locking configuration, closely surrounds at least all or part of the conduit and the locking end part, to obtain rotational locking together of the base and connector.

In addition, the top wall comprises an inner surface defining a hollow housing formed in a thickness of the top wall so that, in the locking configuration, the hollow housing via snap-fit closely receives all or part of the upper surface of the connector body.

The cap body is designed so that, under the effect of the elastic properties of the cap body, the upper surface of the connector body enters into the housing after the first passageway opening surrounds all or part of the conduit and locking end part.

In a first embodiment, the cap body has an L-shaped cross-section.

In one implementation of the first embodiment, the top wall has an outer surface comprising a cantilevered snap-release portion protruding outwardly from a front end of the top wall and which, in the locking configuration, is configured to be manually tilted under the effect of a force applied onto a bottom surface of the snap-release portion to cause release of the upper surface of the connector body from the housing.

In a second embodiment, the width of the first passageway opening, in the non-deformed state thereof, is smaller than a width of the central part of the base.

In a third embodiment, the first vertical wall is configured so that, in the locking configuration, a portion of the locking end part protrudes over a predetermined length beyond a back face of the first passageway opening.

In a fourth embodiment, the top wall and the first vertical wall are in one piece.

In a fifth embodiment, the cap additionally comprises a second vertical wall supporting the top wall, and it is connected at a front end of the top wall so that the cap body has a U-shaped cross-section, the second vertical wall comprising a second passageway opening extending over all or part of a width and/or height of the second vertical wall, and which:
in the locking configuration, closely surrounds at least the end part of the base opposite the locking end part, called opposite end, to obtain further rotational locking together of the base and connector.

Also, the cap body is arranged so that the second passageway opening is only able to surround the opposite end part after the upper surface of the connector body enters the housing.

In a first implementation of the fifth embodiment, the width of the second passageway opening, in the non-deformed state thereof, is smaller than a width of the central part of the base.

In a second implementation of the fifth embodiment, the top wall, the first vertical wall and second vertical wall are in one piece.

In a third implementation of the fifth embodiment, the second vertical wall is configured so that, in the locking configuration, a portion of the opposite end part protrudes over a predetermined length beyond the front face of the second passageway opening.

### BRIEF DESCRIPTION OF THE DRAWINGS

Other characteristics and advantages of the invention will be better understood on reading the following description with reference to the appended drawings given for illustration purposes and not in any respect limiting.

FIG. 1 illustrates a locking assembly of an enteral feeding system.

FIG. 2 gives a cross-sectional view of a cap of the invention.

FIG. 3 is a front view of the cap in [FIG. 2].

FIG. 4 is an overhead view of the cap in [FIG. 2].

FIG. 5 is an underside view of the cap in [FIG. 2].

FIG. 6 illustrates the cap in [FIG. 2] in the engaging configuration.

FIG. 7 illustrates the cap in [FIG. 2] in the course of entering the locking configuration.

FIG. 8 illustrates the cap in [FIG. 2] in the locking configuration.

FIG. 9 illustrates one particular implementation of a cap of the invention.

The Figures are not necessarily drawn to scale, in particular with regard to thickness, for clarity of illustration.

### DESCRIPTION OF EMBODIMENTS

The context in which the invention is implemented is that of a locking assembly for enteral feeding system.

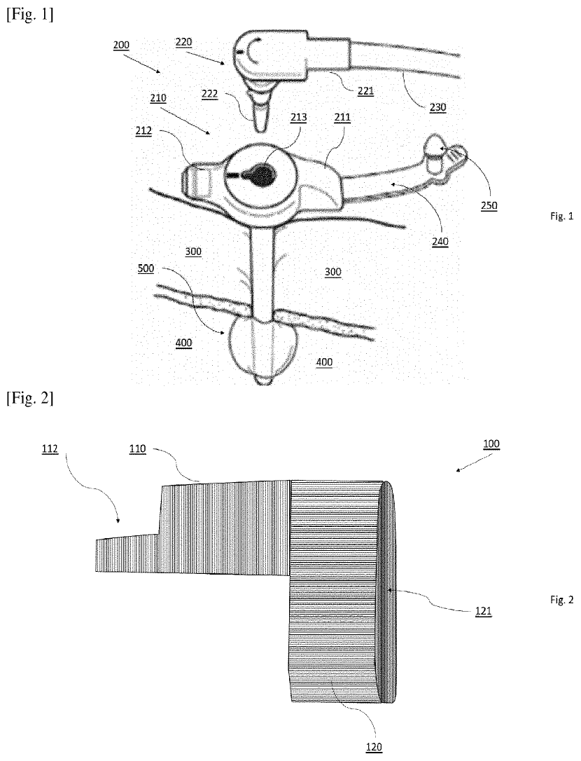

In the example in [FIG. 1], the enteral feeding system is a gastrostomy feeding system.

However, according to needs, it can be envisaged to use other surgical openings into a hollow organ from the skin surface of a wearer of an enteral feeding system, without the need for substantial modifications of the invention.

In the example in [FIG. 1] and in known manner, the enteral feeding system is placed through part of the abdominal wall of a wearer of an enteral feeing system by means of a catheter device which passes through a skin layer 300 of the wearer, then enters the stomach 400 before leading onto an inflatable balloon 500.

As illustrated in [FIG. 1] the enteral feeding system includes a locking assembly 200 comprising a base 210 of the feeding device and a connector 220.

In particular, the base 210 comprises a first end part 211, a central part and a second end part 212.

In practice, the central part is arranged between the first end part 211 and second end part 212.

Also, the first end part 211 has a first end part width, the central part has a central part width and the second end part has a second end part width.

In one example, the central part width is greater than the first end part width and also greater than the second end part width.

In manner known per se, the second end part 212 is positioned at the inflation port of the balloon 500.

Also, the base 210 has an upper surface and a lower surface

By “upper” and “lower” it is meant the parts lying respectively at the top and bottom in the illustration in [FIG. 1].

In particular, the upper surface comprises a first locking element 213.

In the context of the invention, the first locking element 213 is positioned at the feeding port of the enteral feeding system.

Therefore, in one example of the base 210, the base comprises a tab 240 connected to the first end part 211.

The tab 240 comprises a cover 250 configured to close the feeding port in a first position and to open the port in a second position.

As illustrated in [FIG. 1] the connector 220 comprises a connector body and a conduit 221.

The conduit 221 particularly defines a fluid pathway and is coupled to a tube.

In one example, the tube 230 is a medical fluid feeding tube.

Also, the connector body comprises an upper surface and lower surface.

The lower surface of the connector body particularly comprises a second locking element 222 having a shape mating with that of the first locking element 213.

The second locking element 222 is configured to change over between an engaging configuration and a locking configuration.

In the engaging configuration, the first locking element 213 and second locking element 222 engage with each other.

Therefore, in the engaging configuration the locking assembly 200 is in unlocked state.

In a first example of the locking assembly 200, the first locking element 213 comprises a locking opening and the second locking element 222 comprises a locking pin. In this case, in the engaging configuration, the locking opening receives the locking pin.

In a second example of the locking assembly 200, the first locking element 213 comprises a locking pin and the second locking element 222 comprises a locking opening. In this case, in the engaging configuration, the locking opening receives the locking pin.

In locking configuration, in response to rotation of the connector 220 relative to the base 210 in a predetermined locking direction, the conduit 221 lies above an end part 211, 212 of the base 210, called locking end part.

The first locking element 213 and second locking element 222 are engaged with each other so that the connector 220 and base 210 are locked together in vertical and horizontal direction.

By “vertical direction” and “horizontal direction” it is meant the directions respectively lying perpendicular and parallel to the wearer's skin, when the enteral feeding system is in position.

In practice, the locking of the connector 220 with the base 210 is such that it forms a groove formed by an outwardly opening peripheral gap extending between the lower surface of the connector body and the upper surface of the base 210.

Therefore, in the engaging configuration, the locking assembly 200 is in locked state.

In this case, the only means of changing over from the locked state to the unlocked state is to rotate the connector 220 relative to the base 210 in a direction opposite the predetermined locking direction.

Yet some wearers of enteral feeding systems, children for example, frequently move around; especially at night. In this case, the numerous movements made by the wearer of the enteral feeding system can accidentally cause the locking assembly 200 to change over from the locked state to the unlocked state.

Said situation can have serious health consequences for the wearer of an enteral feeding system.

It is one of the objectives of this invention to provide a mechanism which allows preventing of the above-described situation.

For this purpose the inventor proposes a cap which, when the locking assembly 200 is in the locked state, allows rotational locking of the locking assembly 200.

In this manner, the movements made by the wearer of the enteral feeding system are no longer able accidentally to cause changeover of the locking assembly 200 from the locked state to the unlocked state.

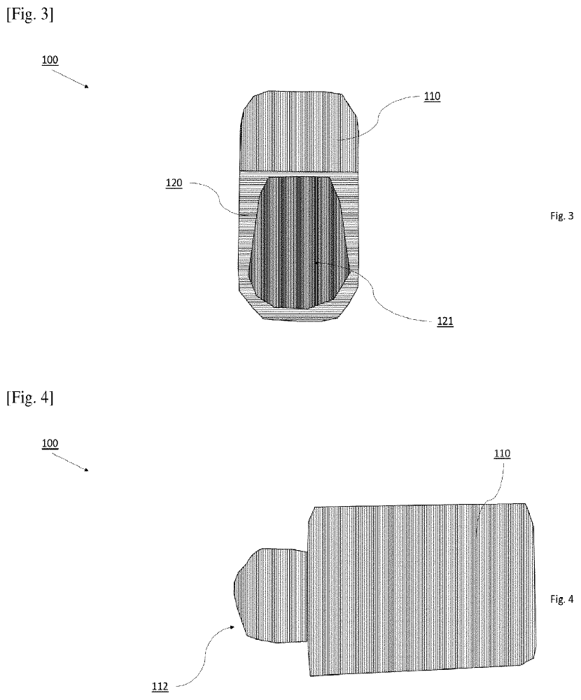

The invention therefore concerns a cap as illustrated in FIGS. 2 to 8.

In practice, the cap 100 comprises a cap body in elastically deformable material.

In practice, this means that the cap body is configured to deform under a force and to return to an initial non-deformed state when the force is removed.

In one particular implementation of the cap body, the elastically deformable material is a material chosen from among: very low density polyethylene, elastomers of propylene/ethylene copolymers, polyether block amides, polyvinyls, ethylene-propylene-diene terpolymers (EPDM), styrene-butadiene-styrene block copolymers (SBS), styrene-ethylene-butadiene-styrene block copolymers (SEBS-SIS), thermoplastic polyurethanes, mixtures of polypropylene with one of the elastomers chosen from among styrene-ethylene-butadiene-styrene block copolymers (SEBS-SIS), ethylene-propylene-diene terpolymers (EPDM), styrene-butadiene-styrene block copolymers (SBS), natural rubbers, nitrile-butyl- or medical silicone elastomers, and any combinations thereof.

However, according to needs, it could be envisaged to use other types of elastically deformable materials, without any substantial modifications to the invention being necessary.

Also, the cap body comprises a cap body having a top wall 110 and a first vertical wall 120.

The top wall 110 extends between a front end and a back end in longitudinal direction.

By “front” and “back” it is meant the parts positioned on the left and right respectively in the illustration in [FIG. 2].

In addition, the first vertical wall 120 supports the top wall 110 and is connected at the back end of the top wall 110.

Also, the first vertical wall 120 has a front face, back face, a thickness, width, and height.

In one particular implementation of the cap body, it has an L-shaped cross-section as illustrated in [FIG. 2].

In a first example of this particular implementation, the top wall 110 and the first vertical wall 120 are in one piece.

In a second example of this implementation, the top wall 110 and the first vertical wall 120 are formed of several assembled parts.

Returning to [FIG. 2], the first vertical wall 120 comprises a first passageway opening 121 extending over all or part of the width and/or height of the first vertical wall 120.

In practice, the first passageway opening 121 leads towards the front face and back face of the first vertical wall 120.

In one particular implementation, the width of the first passageway opening 121 is greater than the outer width of the conduit 221 an also greater than the outer diameter of the tube 230.

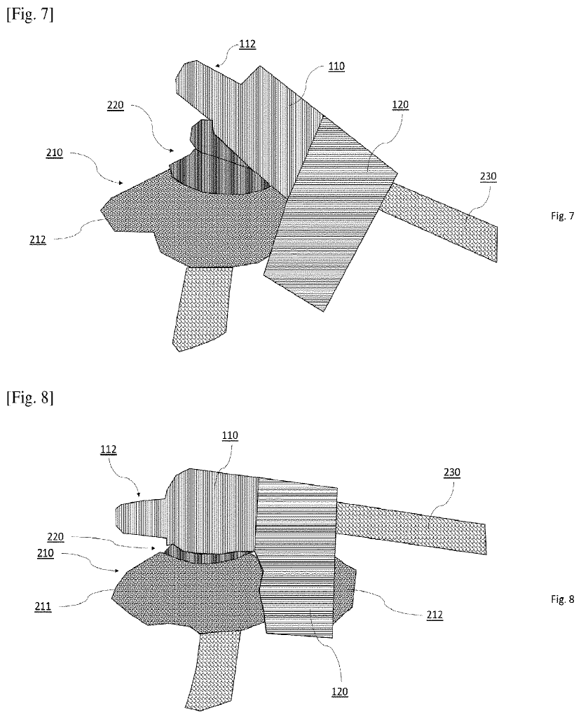

In the engaging configuration illustrated in [FIG. 6], the first passageway opening 121 is intended to be used to pass the tube 230.

In practice, the first passageway opening 121 is configured so that it is possible to pass the connector body from the back face of the first vertical wall 120 through the first passageway opening 121 as far as the front face of the first vertical wall 120, with the result that the tube 230 passes through the first passageway opening 121.

Therefore, as illustrated in [FIG. 6], if the cap body is held in position, it is sufficient to apply a force one or more times on the connector body to move the connector body in a first threading direction until the tube 230 passes through the first passageway opening 121.

In the locking configuration illustrated in FIGS. 7 and 8, the first passageway opening 121 is intended closely to surround at least all or part of the conduit 221 and the locking end part, to obtain rotational locking together of the base 210 and connector 220.

In practice, if the connector body is held in position, it is sufficient to apply a force one or more times on the first vertical wall 120 to move the first vertical wall 120 in a second threading direction opposite the first threading direction, until the first passageway opening 121 closely surrounds all or part of the conduit 221 and locking end part, in particular under the effect of the elastic properties of the cap body.

To ensure proper positioning of the first passageway opening 121 in relation to the conduit 221 and locking end part, provision can be made so that the front face of the first vertical wall 120 comes to abut at least part of the base 210.

In one example, the width of the first passageway opening 121, in the non-deformed state thereof, is smaller than the width of the central part of the base 210.

Therefore, when moving the first vertical wall 120 in the second threading position, the first vertical wall 120 will come to abut the central part of the base to indicate proper positioning of the first passageway opening 121 in relation to the conduit 221 and locking end part.

To reinforce prohibited rotational locking, provision can be made so that a portion of the locking end part protrudes beyond the back face of the first vertical wall 120.

In one example illustrated in [FIG. 8], the first vertical wall 120 is configured so that, in the locking configuration, a portion of the locking end part protrudes over a predetermined length beyond the back face of the first passageway opening 121.

In this example, the thickness of the first vertical wall 120 is substantially narrower than the length of the locking end part.

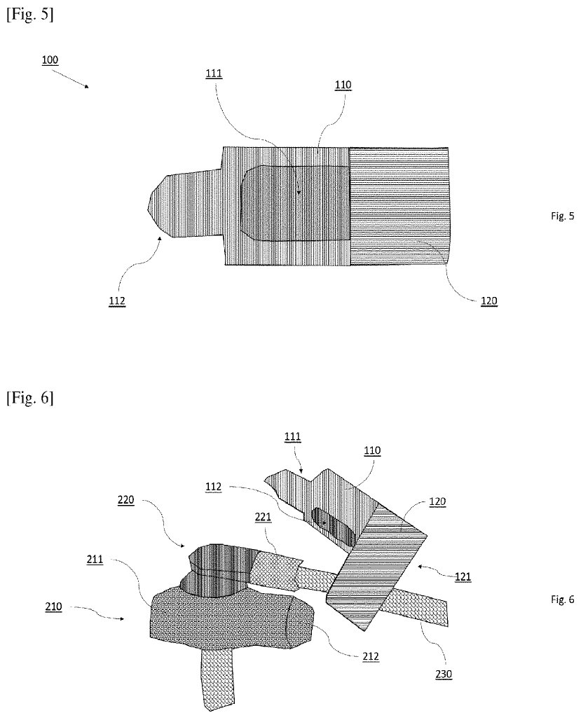

Next, as illustrated in [FIG. 5], the top wall 110 comprises an inner surface defining a hollow housing 111.

In practice in the locking configuration, the housing 111 via snap-fit is intended closely to receive all or part of the upper surface of the connector body, in particular under the effect of the elastic properties of the cap body.

In particular, all or part of an edge of the housing 111 is configured to be fixed via snap-fit into the groove formed by the peripheral gap described above.

Finally, it is noted that the cap body is arranged so that the upper surface of the connector body is only able to enter the housing 111 after the first passageway opening 121 surrounds all or part of the conduit 221 and locking end part.

In a first embodiment of the invention illustrated in FIGS. 2 to 8, the top wall 110 has an outer surface comprising a cantilevered snap-release portion 112 protruding outwardly from the front end of the top wall 110.

Additionally, the snap-release portion 112 has a top surface and bottom surface,

Also, in the locking configuration, the top wall 110 is configured to be manually tilted under the effect of a force applied onto the bottom surface of the snap-release portion 112, to cause release of the upper surface of the connector body from the housing 111.

In particular, snap releasing allows dislodging of the edge of the housing 111 from the groove of the peripheral gap.

In a second embodiment of the invention, it is considered that the first end part 211 and second end part 212 lie opposite each other.

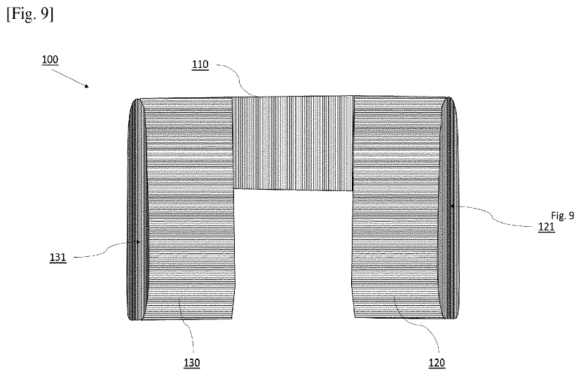

Therefore, as illustrated in [FIG. 9], the cap 100 additionally comprises a second vertical wall 130 supporting the top wall 110 and is connected and the front end of the top wall 110 so that the cap body has a U-shaped cross-section.

In a first example of the second embodiment of the invention, the top wall 110, first vertical wall 120 and second vertical wall 130 are in one piece.

In a second example of the second embodiment of the invention, the top wall 110, first vertical wall 120 and second vertical wall 130 are formed of several assembled parts.

Also, the second vertical wall 130 has a front face, back face, a thickness, width, and height.

Additionally, the second vertical wall 130 comprises a second passageway opening 131 extending over all or part of the width and/or height of the second vertical wall 130.

In practice, the second passageway opening 131 leads onto the front face and back face of the second vertical wall 130.

In one particular embodiment, the width of the second passageway opening 131 is greater than the outer width of the tab 240 and also greater than the outer width of the cover 250.

In the engaging configuration, the second passageway opening 131 is intended for passing of the tab 240.

In practice, the second passageway opening 131 is configured so that the tab 240 can be passed from the back face of the second vertical wall 130 through the second passageway opening 131 as far as the front face of the second vertical wall 130, with the result that the tab 240 passes through the second passageway opening 131.

Therefore, if the cap body is held in position, it is sufficient to apply a force one or more times onto the tab 240 to move the tab 240 through the second passageway opening 131.

Also, in the locking configuration, the second passageway opening 131 is intended closely to surround at least the end part 211, 212 of the base 210 lying opposite the locking end part, called opposite end, to obtain further rotational locking of the base 210 with the connector 220.

In practice, if the connector body is held in position, it is sufficient to apply a force one or more times onto the second vertical wall 130 to move the second vertical wall 130 in the direction of the connector body and base 210 until the second passageway opening 131 closely surrounds at least the opposite end part, in particular under the effect of the elastic properties of the cap body.

Finally, it will be noted that the cap body is arranged so that the second passageway opening 131 is only able to surround the opposite end part after the upper surface of the connector body enters the housing 111.

To ensure proper positioning of the second passageway opening 131 in relation to the opposite end part, provision can be made so that the back face of the second vertical wall 130 comes to abut at least part of the base 210.

In one example, the width of the second passageway opening 131, in the non-deformed state thereof, is smaller than the width of the central part of the base 210.

Therefore, when the second vertical wall 130 is moved in the direction of the connector body and base 210, the second vertical wall 130 will come to abut the central part of the base to indicate proper positioning of the second passageway opening 131 in relation to the opposite end part.

To reinforce prohibited rotational locking, provision can be made so that a portion of the opposite end part protrudes beyond the front face of the second vertical wall 130.

In one example, the second vertical wall 130 is configured so that, in the locking configuration, a portion of the opposite end part protrudes over a predetermined length beyond the front face of the second passageway opening 131.

In this example, the thickness of the second vertical wall 130 is substantially narrower than the length of the opposite end part.

The invention has been described and illustrated. However, the invention is not limited to the disclosed embodiments. Numerous combinations of the variants, alternatives, embodiments, and implementations can be envisaged, without requiring substantial modifications to the invention. A person skilled in the art is able to infer other variants, alternatives, embodiments, and implementations on reading the description and appended Figures, as a function of economic, ergonomic, dimensional restraints to be heeded.

The invention can be the subject of numerous variants and applications other than those described above. In particular, unless otherwise stated, the different structural and functional characteristics of each particular implementation described above are not to be construed as being combined and/or closely and/or inextricably related to each other, but on the contrary as mere juxtapositions. Also, the structural and/or functional characteristics of the different embodiments described above can be the subject in full or in part of any different juxtaposition or any different combination.

1. A cap for a locking assembly of an enteral feeding system, the locking assembly comprising a base of a catheter device and a connector adapted to be removably connected to the base so that the connector and the base are locked with each other in vertical and horizontal directions, the locking assembly being configured to be locked/unlocked by rotation of the connector relative to the base,
the base having a first end part, a central part and a second end part opposite the first end part, the first end part and the second end part protruding from the central part, the base having an upper surface and lower surface, the upper surface comprising a first locking element,
the connector comprising a connector body and a conduit, the conduit defining a fluid pathway and being coupled to a tube, the connector body comprising an upper surface and lower surface, the lower surface comprising a second locking element having a shape mating with the shape of the first locking element and being configured to move between an engaging configuration and a locking configuration,
wherein in the engaging configuration, the first locking element and second locking element engage with each other,
wherein in the locking configuration, in response to rotation of the connector relative to the base in a predetermined locking direction, the conduit lies above a locking end part of the base, and the first locking element and the second locking element are engaged with each other to obtain locking together of the connector and base in vertical and horizontal directions,
wherein:
the cap comprises a cap body in elastically deformable material so that it can deform under a force and return to an initial non-deformed state after removal of the force, the cap body comprising a cap body having a top wall and a first vertical wall supporting the top wall and which is connected at a back end of the top wall,
the first vertical wall comprising a first passageway opening extending over all or part of a width and/or a height of the first vertical wall, and which
in the engaging configuration, allows the passing of a tube,
in the locking configuration, closely surrounds at least all or part of the conduit and the locking end part, to obtain rotational locking together of the base and connector,
the top wall comprising an inner surface defining a hollow housing formed in a thickness of the top wall so that, in the locking configuration, the hollow housing via snap-fit closely receives all or part of the upper surface of the connector body,
the cap body being arranged so that, under the effect of the elastic properties of the cap body, the upper surface of the connector body enters the housing after the first passageway opening surrounds all or part of the conduit and locking end part.

2. The cap according to claim 1, wherein the cap body has an L-shaped cross-section.

3. The cap according to claim 2, wherein the top wall has an outer surface comprising a cantilevered snap-release portion protruding outwardly from a front end of the top wall and which, in the locking configuration, is configured to be manually tilted under the effect of a force applied to a bottom surface of the snap-release portion to release the upper surface of the connector body from the housing.

4. The cap according to claim 1, wherein the width of the first passageway opening, in the non-deformed state thereof, is smaller than a width of the central part of the base.

5. The cap according to claim 1, wherein the first vertical wall is configured so that, in the locking configuration, a portion of the locking end part protrudes over a predetermined length beyond a back face of the first passageway opening.

6. The cap according to claim 1, wherein the top wall and the first vertical wall are in one piece.

7. The cap according to claim 1, additionally comprising a second vertical wall supporting the top wall and which is connected at a front end of the top wall so that the cap body has a U-shaped cross-section, the second vertical wall comprising a second passageway opening extending over all or part of a width and/or height of the second vertical wall, and which:
in the locking configuration, closely surrounds at least an opposite end part of the base opposite the locking end part, to obtain further rotational locking together of the base and connector
the cap body being arranged so that the second passageway opening is only able to surround the opposite end part after the upper surface of the connector body enters the housing.

8. The cap according to claim 7, wherein the width of the second passageway opening, in the non-deformed state thereof, is smaller than a width of the central part of the base.

9. The cap according to claim 7, wherein the top wall, the first vertical wall and the second vertical wall are in one piece.

10. The cap according to claim 7, wherein the second vertical wall is configured so that, in the locking configuration, a portion of the opposite end part protrudes over a predetermined length beyond the front face of the second passageway opening.
